(19) United States
(12) Patent Application Publication
(10) Pub. No.: US 2008/0031872 A1
(43) Pub. Date: Feb. 7, 2008

(54) HIV immunogenic composition and method of administration

(76) Inventor: Nelson M. Karp, Virginia Beach, VA (US)

Correspondence Address: WILLIAMS MULLEN, 222 CENTRAL PARK AVENUE, SUITE 1700, VIRGINIA BEACH, VA, 23462-3035, US

(21) Appl. No.: 11/888,471

(22) Filed: Aug. 1, 2007

Related U.S. Application Data
(60) Provisional application No. 60/835,044, filed on Aug. 2, 2006.

(51) Int. Cl. A61K 39/395; A61K 39/12; A61P 31/00

(52) U.S. Cl. 424/130.1; 424/184.1

(57) ABSTRACT

A method of treatment of HIV by inducing an immune response that includes a robust mucosal immune response. Parenteral administration of an immunogenic composition into the intestinal wall brings the composition into contact with the Peyer's patches of the gut-associated lymphoid system. Administration of multiple immunogenic compositions may track with sites or paths targeted by HIV for infection. The method of administration not only targets the most susceptible part of the host immune system to HIV, but also follows the path of entry and the spread of the naturally occurring disease.

### RELATED APPLICATIONS

This application claims the benefit of U.S. Provisional Application No. 60/835,044 filed Aug. 2, 2006, the disclosure of which is incorporated herein in its entirety.

### FIELD OF THE INVENTION

The present invention relates to an immunogenic composition and a method of administration that induce a robust response in the mucosal compartment of the mammalian immune system against HIV infection.

### BACKGROUND

The need for an effective composition and method of administration directed toward HIV infection is clear. The compositions and methods of administration currently in use are numerous but largely ineffective for many reasons. One reason is that they fail to protect the host from invasion at the virus' targeted points of entry. Still others fail to produce a sufficient immune response in the relevant compartment of the immune system.

Specifically, HIV enters most hosts through the mucosal membranes. Yet most vaccines delivered by injection are not efficient at inducing a mucosal immune response. New research indicates that the mucosal membranes may continue to be a major site of HIV activity, even if drug treatment has reduced HIV count in the peripheral blood. Despite concentrated research efforts and numerous medicaments, HIV continues to spread in both the un-immunized and untreated where it continually mutates and produces new circulating recombinant forms. Clearly a new approach is needed to treat and prevent the continued spread of HIV.

### BRIEF DESCRIPTION

The present method generally comprises parenteral administration of a therapeutically-effective amount of an HIV immunogenic composition into or in the vicinity of the Peyer's patches of the gut-associated lymphoid system to induce a robust mucosal immune response. One embodiment of the present invention additionally tracks with the virus' most common sites of infection: the mucous membranes. In this approach, the present invention not only targets the most susceptible part of the host immune system but also follows the path of entry and spread of the naturally occurring disease. Using these methods of administration of an HIV immunogenic composition offers total body protection and treatment.

The composition and method of the present invention is suitable for the treatment of HIV and HIV-related conditions. The dosage of the compositions and frequency of administration of the compositions will depend on the specifics of the preparations and other clinical factors, such as weight and condition of the host patient or animal and the route of administration. It is to be understood that the present invention has application for both human and veterinary use. It is further understood that other objects, features, and advantages of the present invention will become apparent from the following detailed description. It should be understood, however, that the detailed description and the specific examples, while indicating aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a cross-sectional view of a syringe, useful for the administration of the parenteral component of the immunogenic composition into the host mucosal tissue.

FIG. 2 shows a front view of the syringe with folds and crypts of mucosa shown.

FIG. 3 shows a top view of the syringe and mucosa.

### DETAILED DESCRIPTION OF THE PREFERRED EMBODIMENTS

Eradication of HIV requires an effective treatment and method of administration that will produce a robust response in the host mucosal immune system. Several immunogenic compositions useful for producing a Th-1 and/or Th-2 response may be found in U.S. patent application Ser. Nos. 10/971,199, 10/971,219, 10/971,229, 10/971,426, and 10/971,445, which are hereby incorporated by reference. It should be understood however, that the immunogenic compositions of present invention should not be construed as limited to the immunogenic compositions in the above referenced patents, but also include any effective antigen directed toward HIV. Types of immunogenic composition include whole inactivated; subunit; live attenuated also described as conditionally live vectors; Jennerian; recombinant; and combinations thereof.

For HIV in adults, two primary pathways of transmission have been identified: (1) parenteral (e.g., intravenous drug abuse, transfusion, accidental needle stick); and (2) sexual (e.g., intra-vaginal, intra-rectal, and possibly trans-mucosal involving the male genitalia). Sexual transmission, specifically heterosexual is currently the more prevalent route for spread of the disease in adults. For HIV in children, two pathways of transmission have been identified: (1) parenteral (e.g., skin breaks at time of delivery as well as transfusion related incidents); and (2) through the gastrointestinal system related to nursing.

Additionally, it is known that the immune system of a mammal is roughly divided into four distinct compartments: systemic (tissues and blood), mucosal tissues, body cavities, and skin; each functionally independently. Because of ease of study, most is known about the tissue and blood compartment and its lymphoid tissues, the spleen and lymph nodes. For example, if a proven vaccine is administered by inhalation therapy, it affords the host immune protection primarily in the bronchial associated lymphoid tissue. It is further known that immunologic responses generated on mucosal surfaces usually results in mucosal immunity and minimal, if any, systemic immunity. In contrast, parenteral routes result in systemic immunity with little or no mucosal immunity. See Cecil Czerkinsky, et al., 1999, Mucosal Immunity and Tolerance: Relevance to Vaccine Development, Immunologic Reviews, Vol. 170, pp 197-222, which is hereby incorporated by reference.

The present invention addresses the need for an effective HIV immunogenic composition and delivery method that induces a robust response in the mucosal compartment of the mammalian immune system. The present method generally comprises parenteral administration of a therapeutically-effective amount of an HIV immunogenic composition into the vicinity of the Peyer's patches of the gut-associated lymphoid system. This is achieved by administering the immunogenic composition parenterally into the host's intestinal wall at a position located from the ileum to the terminal rectum.

One embodiment of the present invention additionally tracks with the virus' targeted sites of infection by additional administrations of immunogenic composition to the mucosal sites targeted by the virus for entry into the host. In this approach, the composition and method of administration not only target the most susceptible part of the host immune system, but also follow the path of entry and spread of the naturally occurring disease. Using these methods of administration of an HIV immunogenic composition will provide total body protection and treatment.

“Treating” or “treatment” means the treatment of a disease-state in a mammal, and includes: (a) preventing the disease-state from occurring in a mammal as in a vaccine, and in particular, when such mammal is predisposed to the disease-state but has not yet been diagnosed as having it; (b) inhibiting the disease-state, e.g., arresting its development; and/or (c) relieving the disease-state, e.g., causing regression of the disease-state until a desired endpoint is reached. Treating also includes the amelioration of a symptom of a disease (e.g., lessen the pain or discomfort), wherein such amelioration may or may not be directly affecting the disease (e.g., cause, transmission, expression, etc.)

In one embodiment of the present invention, the parenteral component of the immunogenic composition will target the mucosa-associated lymphoid tissue (or MALT), which is capable of producing a mucosal immune response. Mucosal surfaces are designed for exchange of gasses (lungs), nutrients (digestive tract), sensory functions (eyes, nose, mouth and throat), and reproductive signals (vagina and uterus), so they are more vulnerable to infection than other body surfaces. Research indicates that MALT may be a major site of HIV activity, even if drug treatment has reduced HIV count in the peripheral blood. Therfore targeting this system for an efficient immune response is one of the goals of the present invention.

Another example of MALT is the digestive tract's immune system often referred to as gut-associated lymphoid tissue (GALT). GALT includes the tonsils and adenoids at the back of the throat, the Peyer's patches of the small intestine, the appendix, isolated lymphoid follicles of the large intestine and rectum, and small foci of lymphocytes and plasma cells in the lamina propria of the gut wall.

Peyer's patches facilitate the generation of the immune response within the mucosa. They are aggregations of lymphoid tissue usually found in the lowest portion to the small intestine (ileum) in humans. Peyer's patches are also the primary site of HIV replication, both in initial infections and throughout the course of disease.

Peyer's patches generally lie in the region of the intestinal wall's lamina propria layer of the mucosa and extending into the submucosa. Peyer's patches include microfold cells (or M cells) in the follicle-associated epithelium. The M-cells sample antigens from the lumen of the host's small intestine and by transcytosis deliver the antigen to antigen presenting cells and lymphocytes located in a unique pocket-like structure on their basolateral side. Thus, parenteral administration into the Peyer's patches would specfically produce a GALT immune response.

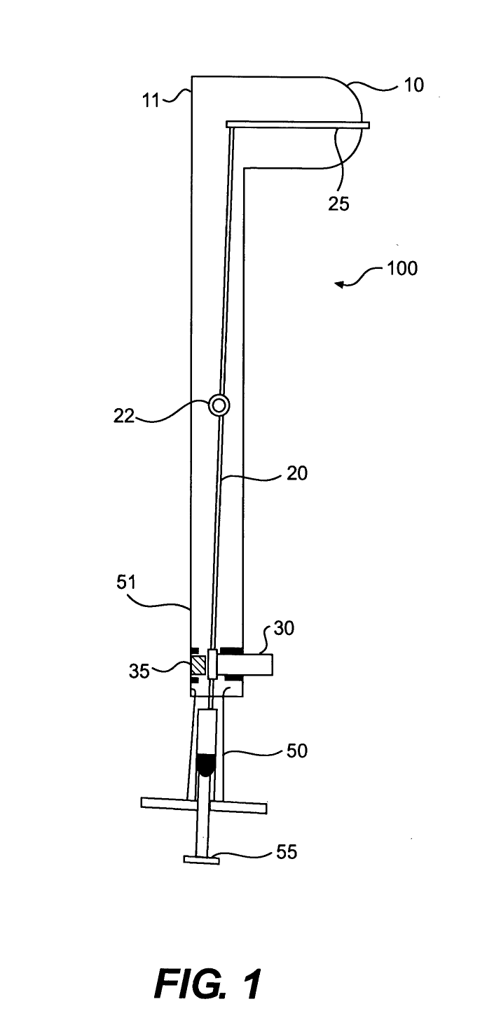

A breach of the mucosal barrier tilts the response towards a Th-1 and/or Th-2 behavior and away from anergy and/or suppression. Therefore, a device is needed that will penetrate the epithelial lining and allow administration of the treatment bypassing the epithelial and reaching M-cells, which form the gateway to MALT. This can be accomplished by a variety of mechanical devices useful in the parenteral administration of immunogenic compositions. For example, parental administration into the host mucosa low in the large colon or rectal area may be undertaken by a device such as that shown in FIG. 1.

FIG. 1 is a cross-sectional view of an embodiment of such a syringe, depicted here as device 100. A broad semi-circular or half oval-head 10 housing needle 25 is disposed at a distal end 11 and is adapted to being pressed against the mucosa 200 (not shown) of the vagina or rectum. In the simple embodiment illustrated for lower colon access, button 30 may be counterbalanced against spring 35, so that pressing button 30 and compressing spring 35 will rotate lever 20 about pivot 22 and expose needle 25. Other mechanisms, such as solenoids, piezoelectric actuators, pressurized injectors, linkages, etc., may also provide the desired function of retracting and exposing needle 25. A sliding syringe assembly 50 is located on proximate end 51 and is in fluid communication with needle 25; during insertion and use, syringe assembly 50 will remain outside the rectum or vagina for operation of plunger 55 and button 30. Alternatively, compressed gas, piezoelectric microdispensing, or other means may be used to drive the vaccine from needle 25 when desired. Deeper access may require incorporation of such an injector into a biocompatible flexible colon access tool similar to, for example, a colonoscope.

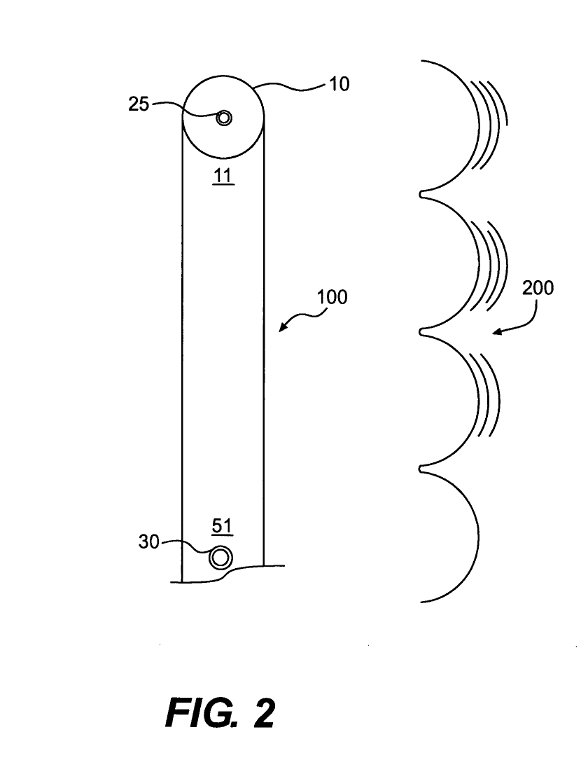

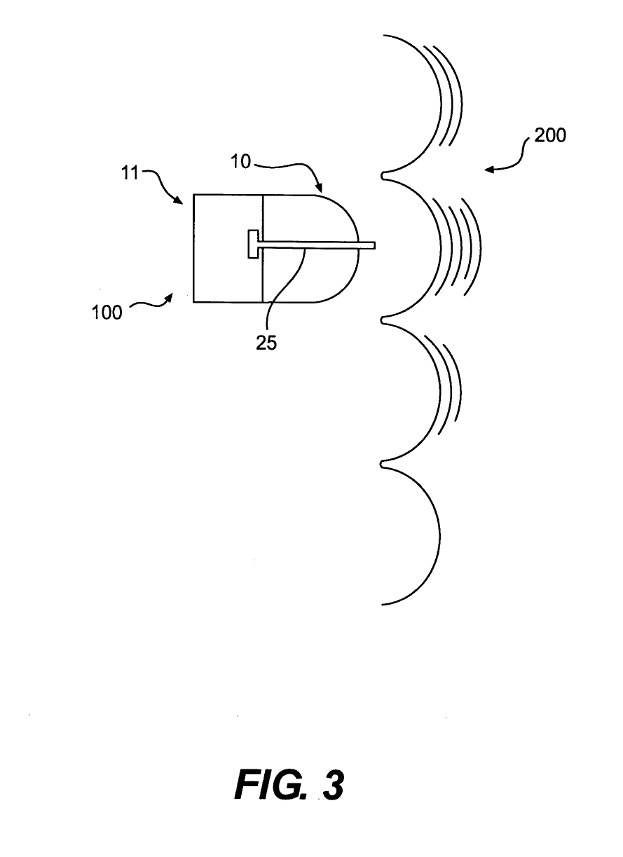

FIG. 2 is a front view of this example of device 100 with folds and crypts of mucosa 200 shown. As may be seen, device 100 overcomes the difficulty of folds or crypts in the rectum and vagina, which otherwise would make it difficult to determine the length of needle necessary to penetrate the cell lining. To be effective, the vaccine must breach the epithelial lining only. Without breaching the epithelial barrier, anergy, tolerance, or immune non-responsiveness will result. An analogy may be drawn to the passage of food through the intestine, which is protected from an immune response by an intact mucosal lining. FIG. 3 is a top view of device 100 and mucosa 200. Thus, device 100 may be used to control the depth of insertion of needle 25, while adapting to the contours of the folds of mucosa 200. Preferably, for inducing a gut-associated lymphoid system immune response via Peyer's patches, such a device would be integrated into a flexible colonoscope or other colon access device for reaching distal portions of the host's ileum.

In one embodiment, the method of parenteral administration of the immunogenic composition is directed to the intestinal wall at a position located from the ileum to the terminal rectum. In another embodiment, the method of parenteral administration of the immunogenic composition is directed to a position located from the ileum to the descending colon. In another embodiment, the method of parenteral administration of the immunogenic composition is directed to the ileum. In a further embodiment, the method of parenteral administration of the immunogenic composition is directed to points within the lamina propria layer of the mucosa extending to the submucosa of the intestinal wall. The method may further comprise using a mechanical device designed to penetrate the epithelial lining thereby avoiding immune reactive pathways in the epithelial and M cells.

As previously mentioned, HIV can be spread orally, and the oral route is a predominant means for the spread of the disease among children in third world countries. An HIV positive woman nursing an HIV negative infant will spread the disease to the infant approximately 35% of the time if the appropriate anti-retroviral treatment is not administered to both the mother and infant. This percentage has been gleaned from data in industrialized countries. In third world countries, the actual percentage of transmission via the route of breast milk is presumed higher because the health of the mother and infant is often compromised by nutritional deficiencies and other concomitant diseases.

The oral transmission of AIDS may also bring light to previously unexplained epidemiologic statistics in third world countries. For example, the United Nations (UN) has stated that the spread of AIDS has exceeded all worst case scenarios provided by UN computer models. Oral transmission of AIDS is not a component of these models. Yet the lack of sanitary conditions could easily expose an HIV negative patient to HIV positive bodily fluids.

As previously mentioned, one embodiment of the present invention additionally tracks administration of immunogenic composition with one or more of the virus' targeted sites of infection. This may be achieved by supplying additional parenteral administrations of an immunogenic composition to the mucosal sites targeted by the virus for entry into the host. These mucosal sites for additional parenteral administrations include any lubricated inner lining of the mouth, nasal passages, vagina and urethra, male genitalia, and rectal, as well as any membrane or lining which contains mucous secreting glands is collectively referred to as mucosa, mucosal tissues, or mucosal lining. Alternatively, additional administrations of the immunogenic composition may be provided to the host orally. By providing additional administrations of immunogenic composition, either parenterally and/or orally, the present invention not only targets the most susceptible part of the host immune system, but also follows the path of entry and spread of the naturally occurring disease. Using these methods of administration of an HIV immunogenic composition will provide total body protection and treatment.

Therefore, in one embodiment, the method of administering the immunogenic composition directed to the intestinal wall at a position located from the ileum to the terminal rectum is combined with administering an immunogenic composition directed to at least one additional mucosal tissue. Alternatively, the method of administering the immunogenic composition directed to the intestinal wall at a position located from the ileum to the terminal rectum is combined with administering an immunogenic composition orally.

Regarding the oral component of the present invention, one question is whether such an oral formulation would provide treatment in the rectum or vagina. Certainly rectally targeted treatment could result if the oral formulation is enteric coated thereby allowing absorption to occur in the terminal ileum and large intestine. The lymphatic system of the trunk, abdominal and pelvic organs, as well as lower extremities, ultimately drains into the thoracic duct which terminates into the subclavian vein on the left. Additionally, the lymphatic drainage of the female reproductive organs crosses anatomical pathways with the lymphatic drainage from the large and small intestine. Therefore, while not intending to be based on any one theory, it is believed that the oral component of the immunogenic composition properly formulated with an enteric coating will induce some immune response in the vaginal as well as rectal mucosal tissues.

With further regard to the oral component of the present invention, studies of oral transmission of HIV in infants has led to knowledge that breast milk contains not only free, non-cell associated HIV particles, but also an abundance of antibodies to various infectious diseases, including HIV. To ensure transport of the HIV particle through the gastrointestinal tract and to protect it from degradation by the harshly acidic, gastric environment, the orally administered immunogenic composition may, in one embodiment, be coated with antibodies. Examples of such antibodies include but are not limited to IgG₁, IgG₂, IgG₃, IgG₄, IgM, IgA, IgD and IgE or derivatives thereof such as Fab fragment.

It is anticipated that the antibodies may facilitate entry of the virus into the endothelial cells lining the gastrointestinal tract. Supporting evidence includes studies showing neutralizing antibodies to HIV enhance the cellular uptake of the virus in T cells, B cells and macrophages. Additionally, the antibodies to HIV found in a mother's breast milk are bound to complement proteins or fragments derived thereof. These complement fragments have also been shown to enhance viral entry into immunologic as well as non-immunologic cells.

Therefore, in one embodiment, an immunogenic composition is associated with complement proteins and protein fragments that facilitate absorption; transport across the endothelial lining of the large intestine; and integrate into a variety of immune cells. Examples of such proteins and protein fragments include, but are not limited to, the complement derived opsonins C3b, C5b, chemotaxic agents C5a and the C5b6,7 complex, and anaphylatoxins C3a and C4a.

Additionally, other studies indicate that lipids may be used to enhance uptake of antigens by mucosal cell membranes. Therefore in another embodiment of the present invention the immunogenic composition is embedded with lipids in therapeutically effective amounts.

In another embodiment, a vaginal suppository (or tampon) and a rectal suppository of the composition could be administered as an adjuvant to the orally and parenterally administered immunogenic composition. Rectal and vaginal absorption of drugs, however, is erratic and unpredictable. The vaginal route can be compromised by menstruation or post-menopausal status. Furthermore, when the host is exposed to proteins or other substances across the mucosal boundary of the rectum or vagina, a compelling problem arises of immuno-suppression mediated by T-suppressor cells and T-regulatory cells.

### Definitions

Mammal and patient include warm blooded mammals (e.g., humans, domesticated animals, and wild animals).

“Therapeutically effective amount” includes an amount of the immunogenic compound, protein, protein fragments, antibodies, complement fragments and combinations thereof of the present invention that is effective when administered alone or in combination to treat an indication listed herein. “Therapeutically effective amount” also includes an amount of the combination of compounds claimed that is effective to treat the desired indication. The combination of compounds can be a synergistic combination. Synergy, as described, for example, by Chou and Talalay, Adv. Enzyme Regul. 1984, 22:27-55, which is hereby incorporated, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased effect, or some other beneficial effect of the combination compared with the individual components.

### Dosage and Formulation

The compounds of the present invention can be formulated as pharmaceutical compositions and administered to a mammalian host, such as a human patient in a variety of forms adapted to the chosen route of administration, e.g., orally or parenterally, by intravenous, intramuscular, topical, inhalation, subcutaneous routes, etc. as generally understood in the art. Exemplary pharmaceutical compositions are disclosed in “Remington: The Science and Practice of Pharmacy,” A. Gennaro, ed., 20th edition, Lippincott, Williams & Wilkins, Philadelphia, Pa.

The present compounds may be administered, e.g., orally, in combination with a pharmaceutically acceptable carrier such as an inert diluent or an assimilable edible carrier. They may be enclosed in hard or soft shell gelatin capsules, may be compressed into tablets or may be incorporated directly with the food of the patient's diet. For oral therapeutic administration, the active compound may be combined with one or more carriers and used in the form of ingestible tablets, buccal tablets, troches, capsules, elixirs, suspensions, syrups, wafers, and the like. The amount of active compound in such therapeutically useful compositions is such that an effective dosage level will be obtained.

The tablets, troches, pills, capsules, and the like may also contain the following: binders such as gum tragacanth, acacia, corn starch or gelatin; carriers such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. It will be understood that any material used in preparing any unit dosage form should be pharmaceutically acceptable and substantially non-toxic in the amounts employed. In addition, the active compound may be incorporated into sustained-release preparations and devices.

The active compound may also be administered parenterally e.g., intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for parenteral administration can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may be desirable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as elected, followed by filter sterilization. In the case of sterile powders for the preparation of sterile injectable solutions, desirable methods of preparation include vacuum drying and freeze drying techniques, which yield a powder of the active ingredient plus any additional desired ingredient present in the previously sterile-filtered solutions.

The amount of the compound or an active salt or derivative thereof required for use in treatment will vary not only with the particular compound or salt selected but also with the route of administration, the nature of the condition being treated, and the age and condition of the patient and will be ultimately at the discretion of the attendant physician or clinician.

Whatever dose or route of administration is intended, a Th-1 response with HIV disease is most attractive. To facilitate this, a short half-life of the administered immunogen would be most advantageous. Furthermore, the minimal dosage necessary to elicit an immune response is most likely to result in a Th-1 bias. Large doses of immunogen can skew the immune system into a Th-2 direction or anergy, both of which are counter productive. With HIV disease, a Th-2 response often develops, in large part, due to the chronicity of the infection.

The compounds or compositions of the invention can also be administered by inhalation from an inhaler, insufflator, atomizer or pressurized pack or other means of delivering an aerosol spray. Pressurized packs may comprise a suitable propellant such as carbon dioxide or other suitable gas. In case of a pressurized aerosol, the dosage unit may be determined by providing a value to deliver a metered amount. The inhalers, insufflators, atomizers are fully described in pharmaceutical reference books such as Remington's Pharmaceutical Sciences, Volumes 16 (1980) or 18 (1990) (Mack Publishing Co.).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple pills or by application of a plurality of injections into the mucosal tissue.

All patents, patent applications, books and literature cited in the specification are hereby incorporated by reference in their entirety. In the case of any inconsistencies, the present disclosure including any definitions therein will prevail.

The invention has been described with reference to various specific and detailed aspects and techniques. However, it should be understood that many variations and modifications may be made while remaining within the spirit and scope of the invention. The present invention, thus generally described, will be understood more readily by reference to the above examples, which are provided by way of illustration and are not intended to be limiting of the present invention.

It is therefore to be understood that within the scope of the appended claims, the invention may be practiced otherwise that as specifically described herein. Additionally, any aspect or feature of the present invention may be combined with any other aspect or feature of the invention.

In conclusion, the present invention addresses the need for an effective HIV immunogenic composition and delivery method that induces a robust response in the mucosal compartment of the mammalian immune system. The present method generally comprises parenteral administration of a therapeutically-effective amount of an HIV immunogenic composition to induce a robust mucosal immune response by targeting the Peyer's patches of the gut-associated lymphoid system. One embodiment of the present invention additionally tracks with the virus' targeted sites of infection. In this approach, the composition and method of administration not only target the most susceptible part of the host immune system but also follow the path of entry and spread of the naturally occurring disease. Using these methods of administration of an HIV immunogenic composition will provide total body protection and treatment.

What is claimed is:

1. A method of treatment of HIV by inducing an immune response that includes a mucosal immune response, comprising the steps of:
selecting and providing a therapeutically effective amount of a first immunogenic composition for parenteral administration to produce a gut-associated lymphoid system immune response; and
administering the first immunogenic composition parenterally to the intestinal wall at a position located from the ileum to the terminal rectum.

2. The method of claim 1, wherein said parenteral administration of the first immunogenic composition is directed to a position located from the ileum to the descending colon.

3. The method of claim 1, wherein said parenteral administration of the first immunogenic composition is directed to the ileum.

4. The method of claim 1, wherein the step of administering the first immunogenic composition parenterally is directed to points within the lamina propria layer of the mucosa extending to the submucosa of the intestinal wall.

5. The method of claim 1, wherein the step of administering the first immunogenic composition further comprises using a mechanical device designed to penetrate the epithelial lining thereby avoiding immune reactive pathways in the epithelial and M cells.

6. The method of claim 1, further comprising the steps of:
selecting and providing a therapeutically effective amount of at least one second immunogenic composition for administration to the mucosal tissues to produce a mucosal immune response; and
administering the second immunogenic composition to the mucosal tissues.

7. The method of claim 1, further comprising the steps:
selecting and providing a therapeutically effective amount of a second immunogenic composition for parenteral administration to the mucosal tissues to produce a mucosal immune response; and
administering the second immunogenic composition parenterally to the mucosal tissues.

8. The method of claim 1, further comprising the steps:
selecting and providing a therapeutically effective amount of a second immunogenic composition for oral administration to produce a mucosal immune response; and
administering the second immunogenic composition orally.

9. The method of claim 1, further comprising the steps of:
selecting and providing a therapeutically effective amount of a second immunogenic composition for oral administration to produce a mucosal immune response; and
selecting and providing a carrier pharmaceutically acceptable in oral administration for use with the second immunogenic composition;
administering orally the second immunogenic composition in combination with the carrier pharmaceutically acceptable in oral administration.

10. The method of claim 1, wherein said first immunogenic composition is formulated in combination with a carrier pharmaceutically acceptable in parenteral administration for use with the first immunogenic composition.

11. The method of claim 1, further comprising the steps of:
selecting and providing a therapeutically effective amount of a second immunogenic composition, in combination with an enteric coating to facilitate absorption for oral administration to produce a mucosal immune response; and
administering the enteric coated second immunogenic composition orally.

12. The method of claim 1, further comprising the steps of:
selecting and providing a therapeutically effective amount of a second immunogenic composition, in combination with a coating of at least one antibody selected from the group, IgG₁, IgG₂, IgG₃, IgG₄, IgM, IgA, IgD, IgE, breast milk HIV antibodies, or derivatives thereof, to facilitate absorption, for oral administration to produce a mucosal immune response; and
administering the second immunogenic composition and the at least one antibody orally.

13. The method of claim 1, further comprising the steps of:
selecting and providing a therapeutically effective amount of a second immunogenic composition, in combination with at least one protein selected from the group, C3b, C5b, C5a, C5b6,7 complex, C3a, C4a, or fragments thereof, to facilitate absorption, for oral administration to produce a mucosal immune response; and
administering the second immunogenic composition and the at least one protein orally.

14. The method of claim 1, further comprising the step of administering at least one pharmaceutically acceptable lipid to facilitate absorption.

15. The method of claim 1, further comprising the step of administering at least one pharmaceutically acceptable adjuvant.

16. The method of claim 1, further comprising the step of administering at least one pharmaceutically acceptable adjuvant administered to the mucosal tissues.

17. The method of claim 1, further comprising the step of administering at least one pharmaceutically acceptable adjuvant, wherein the adjuvant is administered orally, vaginally, rectally, ureterally, or parenterally to the intestinal wall.
